(19) United States
(12) Patent Application Publication
(10) Pub. No.: US 2009/0024040 A1
(43) Pub. Date: Jan. 22, 2009

(54) Wall-Contacting Intravascular Ultrasound Probe Catheters

(75) Inventor: Eduardo Ignacio Cespedes, Folsom, CA (US)

Correspondence Address: PATTON BOGGS LLP, 8484 WESTPARK DRIVE, SUITE 900, MCLEAN, VA, 22102, US

(73) Assignee: Prescient Medical, Inc., Doylestown, PA (US)

(21) Appl. No.: 12/137,227

(22) Filed: Jun. 11, 2008

Related U.S. Application Data
(60) Provisional application No. 60/950,922, filed on Jul. 20, 2007.

(51) Int. Cl. A61B 8/12

(52) U.S. Cl. 600/467

(57) ABSTRACT

The present invention provides intravascular diagnostic catheters that include one or more wall-contacting/wall-approaching probes including IVUS probe elements and diagnostic systems including such catheters, for the evaluation and diagnosis of blood vessels. Also provided are intravascular catheters in which the wall-contacting/wall-approaching probes further include an optical probe element and systems including such catheters, for combined IVUS and optical analysis of a blood vessel wall.

### CROSS-REFERENCE TO RELATED APPLICATIONS

This application claims the benefit of U.S. provisional application Ser. No. 60/950,922 filed Jul. 20, 2007, which is incorporated by reference herein in its entirety.

### FIELD OF THE INVENTION

The invention relates generally to the fields of catheter-based intravascular ultrasound (IVUS) and intravascular optical spectroscopy.

### BACKGROUND OF INVENTION

Various modalities for diagnostically interrogating blood vessel walls to locate and characterize atherosclerotic lesions have been previously proposed including intravascular ultrasound (IVUS) and optical spectroscopic techniques, such as Raman spectroscopy. IVUS catheters have generally fallen into two categories: a single transducer that is rotated about a central axis or an array of elements that are phased (controlled delays) relative to one another on excitation or collection to provide spatial information. Piezoelectric effect-based ultrasound detectors are well known in the art. More recently, optics-based ultrasound sensors, such as Fabry-Perot interferometers, have also been described.

The following patents and publications are also background to the present invention.

U.S. Pat. No. 5,840,023 discloses systems and methods of acoustic imaging for medical diagnosis, and is incorporated by reference herein in its entirety.

U.S. Pat. No. 6,277,077 discloses a basket-style cardiac mapping catheter having basket arms that include ultrasound transducers and mapping electrodes, and is incorporated by reference herein in its entirety.

U.S. Pat. No. 6,281,976 discloses fiber-optic Fabry-Perot interferometer sensors and methods of measurement therewith, and is incorporated by reference herein in its entirety.

U.S. Pat. No. 6,445,939 discloses ultra-small optical probes that include an optical fiber and a lens that has at least substantially the same diameter as the fiber and which may be in communication with a beam director, and is incorporated by reference herein in its entirety.

U.S. Pat. No. 6,522,913 discloses systems and methods for visualizing tissue during diagnostic or therapeutic procedures that utilize a support structure that brings sensors into contact with the lumen wall of a blood vessel, and is incorporated by reference herein in its entirety.

U.S. Pat. No. 6,701,181 discloses multi-path optical catheters, and is incorporated by reference herein in its entirety.

U.S. Pat. No. 6,813,401 discloses methods for fabricating Fabry-Perot polymer film sensing interferometers on optical fiber substrates, and is incorporated by reference herein in its entirety.

U.S. Pat. No. 6,873,868 discloses multi-fiber catheter probe arrangements for tissue analysis or treatment, and is incorporated by reference herein in its entirety.

U.S. Pat. No. 6,839,496 discloses optical fiber probes for photoacoustic material analysis, and is incorporated by reference herein in its entirety.

U.S. Pat. No. 6,949,072 discloses devices for vulnerable plaque detection that combine IVUS and optical analysis, and is incorporated by reference herein in its entirety.

U.S. Publication No. 2002/0183622 discloses a fiber-optic apparatus and method for the optical imaging of tissue samples, and is incorporated by reference herein in its entirety.

U.S. Publication No. 2003/0032880 discloses apparatuses and methods for ultrasonically identifying vulnerable plaques, and is incorporated by reference herein in its entirety.

U.S. Publication No. 2003/0125630 discloses catheter probe arrangements for tissue analysis by radiant energy delivery and radiant energy collection, and is incorporated by reference herein in its entirety.

Each of U.S. Publication Nos. 2003/0199747, 2003/0199767 and 2003/0199768 discloses a basket catheter having a centrally disposed intravascular ultrasound imaging element and peripheral optical thermography sensors on the basket arms, and is incorporated by reference herein in its entirety.

U.S. Publication No. 2004/0260182 discloses intraluminal spectroscope devices with wall-contacting probes, and is incorporated by reference herein in its entirety.

U.S. Publication No. 2005/0054934 discloses an optical catheter with dual-stage beam redirector, and is incorporated by reference herein in its entirety.

U.S. Publication No. 2005/0165315 discloses a side-firing fiber-optic array probe, and is incorporated by reference herein in its entirety.

U.S. Publication No. 2006/0139633 discloses the use of high wavenumber Raman spectroscopy for evaluating tissue, and is incorporated by reference herein in its entirety.

In view of the above, what is needed and desirable are new and improved apparatuses and methods for the intravascular evaluation of blood vessel walls using ultrasound alone or in combination with optical analytical methods.

### SUMMARY OF INVENTION

The present invention provides intravascular diagnostic catheters that include one or more wall contacting probes having wall-contacting/wall-approaching IVUS probe elements for the evaluation and diagnosis of blood vessels.

One embodiment of the invention provides an intravascular catheter including at least one radially extendable wall-contacting/wall-approaching probe element that includes a wall-contacting/wall-approaching portion, which includes both a Raman spectroscopy probe element, such as a front or side viewing optical fiber assembly and an IVUS element such as an ultrasound transducer operating at 10 MHz or above, 20 MHz or above, 30 MHz or above, 40 MHz or above, 50 MHz or above, or 60 MHz or above. The ultrasound transducer may, for example, be a high-frequency ultrasound transducer.

A related embodiment of the invention provides a basket-type intravascular catheter including a basket section that includes at least two radially extendable probe arms, each having a wall-contacting/wall-approaching portion, wherein at least one of the probe arms, for example, all of the probe arms, include both a optical spectroscopy probe element, such as a front or side viewing optical fiber assembly and a IVUS element, such as an ultrasound transducer operating at 10 MHz or above, such as at 20 MHz or above, such as at 40 MHz or above, such as at 60 MHz or above. The ultrasound transducer may, for example, be a high-frequency ultrasound transducer.

The invention also provides methods for evaluating blood vessels using the apparatuses and systems of the invention. One embodiment of the invention provides a method for locating and/or characterizing lipid rich deposits and/or lesions in a blood vessel such as an artery that include interrogating a blood vessel wall by IVUS and an optical analytical technique, such as Raman spectroscopy, using a catheter or catheter system according to the invention. Another embodiment of the invention provides a method for locating and/or characterizing atherosclerotic lesions, such as vulnerable plaques, in a blood vessel that includes using a catheter system according to the invention to interrogate a blood vessel wall. Thus, a catheter system according to the invention may be used to diagnostically interrogate a blood vessel to provide or assist in providing a diagnosis of the blood vessel and/or may be used to provide guidance for application of a local therapy within a blood vessel, such as therapeutic irradiation and/or deployment of a prosthesis such as a stent.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1A shows an embodiment basket-style intravascular catheter comprising radially extendable, wall-contacting/wall-approaching probe arms each of which includes a side-viewing optical element for an optical analytical technique, such as Raman spectroscopy, and an ultrasound transducer for intravascular ultrasound.

FIGS. 1B-E show variations in the cross-sectional detail of a probe arm as shown in the embodiment of FIG. 1A.

FIG. 2 shows an embodiment similar to that of FIG. 1, but further including a centrally disposed, radial scanning IVUS element within the basket section of the catheter.

FIG. 3 shows an embodiment similar to that of FIG. 1, but further including a centrally disposed, radial scanning IVUS element proximal to the basket section of the catheter.

FIG. 4 shows a diagnostic catheter system embodiment of the invention.

FIG. 5 shows Raman spectra of cholesterol and various cholesterol esters in the Raman high wavenumber region.

### DETAILED DESCRIPTION

The present invention provides intravascular diagnostic catheters that include one or more wall contacting probes having wall-contacting/wall-approaching IVUS probe elements for the evaluation and diagnosis of blood vessels.

In one embodiment, the invention provides intravascular diagnostic catheters that include one or more wall contacting probes that have both an optical probe element, such as a Raman spectroscopy probe element, and an IVUS probe element, and methods of use thereof to evaluate and diagnose blood vessels.

Catheter-based intravascular ultrasound (IVUS) has typically been performed using a IVUS imaging element disposed in/on a/the central shaft of an intravascular catheter to provide radial scanning of the vessel wall through a field of blood. Radial scanning, either by mechanical rotation or by use of a phase array, has been used to build up a 360 degree view of the vessel. The present inventors have appreciated that significant information concerning the physical nature of target tissue (structure, composition, depth etc.) in a blood vessel wall may be obtained using wall-contacting/wall-approaching IVUS probes, despite the reduced radial coverage attendant therewith versus conventional radially scanning IVUS. Advantageously, since obscuring blood is no longer an issue, the use of wall-contacting/wall-approaching ultrasound probes according to the present invention allows very high frequency ultrasound transducers to be employed, thereby providing a very high resolution and information content for the tissue region that are interrogated. Where radially-segregated information is desired about a blood vessel, basket-style catheter embodiments including radially separated, wall-contacting/wall-approaching IVUS probe arms may be employed, such as those having 3, 4, 5, 6, 7, 8 or more radially separated probe arms.

Any suitable type of ultrasound transducers may be used in implementing the invention. For example, transducers made from conventional piezoelectric materials may be used and newer types of high speed transducer such as capacitive micromachined ultrasonic transducers (CMUTs) and those made from ceramic-based materials may be used. Optoacoustic stimulation of ultrasound may also be used according to the invention. The IVUS transducer elements used in the catheter embodiments of the invention may, for example, operate at a frequency of at least 10 MHz, for example, in a range of 10 to about 100 MHz, or at a frequency of at least 20 MHz, for example, in a range of 20 MHz to about 100 MHz, or at a frequency of at least 40 MHz, for example, in a range of 40 MHz to about 100 MHz. It should be understood that the frequencies given are center frequencies. Generally, the ultrasound transducer may operate at 10 MHz or above, 20 MHz or above, 30 MHz or above, 40 MHz or above, 50 MHz or above, or 60 MHz or above. The ultrasound transducer may, for example, be a high-frequency ultrasound transducer.

Any suitable sort of side/lateral-viewing optical assembly(ies) may be used to provide a side-viewing optical probe element and numerous sorts of side viewing optics are well known in the art. For example, a 45-deg (or other angle) mirror face or a prism can be used to laterally direct/redirect light from an optical fiber. Similarly, an optical fiber can be provided with an angularly faceted tip to direct and receive light that is off-axis with respect to the fiber. Generally, the optical probe arm(s) of embodiments of the invention will have disposed therein on or more optical fibers forming the optical probe element thereof.

Optical analytical techniques that may be employed in conjunction with IVUS according to the invention include, for example, Raman spectroscopy such as high wavenumber Raman spectroscopy and/or fingerprint region Raman spectroscopy, laser-induced fluorescence spectroscopy (LIFS), such as time-resolved laser-induced fluorescence spectroscopy (TR-LIFS), absorbance spectroscopy, such as infrared (IR) or near infra-red (NIR) absorbance spectroscopy, interferometry such as optical coherence tomography (OCT) and low-coherence interferometry (LCI), and laser speckle spectroscopy. U.S. Publication No. 2006/0139633 discloses methods and systems of high-wavenumber Raman spectroscopy for measuring tissue properties including for characterizing atherosclerotic plaques, and is incorporated by reference herein in its entirety. U.S. Pat. No. 6,272,376 discloses methods and systems of time-resolved laser-induced fluorescence spectroscopy, including for identifying and characterizing lipid-rich vascular lesions, and is incorporated by reference herein in its entirety. International Publication No. WO2005019800 discloses methods for fluorescence lifetime imaging microscopy and spectroscopy, including ultra-fast methods for analysis of fluorescence lifetime imaging is also described, facilitating real-time analysis of compositional and functional changes in samples, and is incorporated by reference herein in its entirety. Low-coherence interferometry methods, such as OCT, are disclosed in U.S. Pat. Nos. 7,190,464, 6,903,854 and 6,134,003 and U.S. Publication No. 2005/0020925, each of which is incorporated by reference herein in its entirety. U.S. Pat. No. 7,061,606 and U.S. Pub. No. 2004/0077950 disclose near-infrared (NIR) spectroscopy, such as analysis of NIR absorbance, transmittance and reflectance spectra, and are incorporated by reference herein in their entireties. U.S. Pub. No. 2002/0183601 discloses laser speckle-based methods and systems for analyzing tissue, and is incorporated by reference herein in its entirety.

Particularly advantageous is the combination of a chemical composition-determining optical technique such as Raman spectroscopy and/or LIFS, especially TR-LIFS, with physical property determination by IVUS in the present invention.

The inventions also provides combined Optical Analysis/IVUS systems that generally include, in addition to a catheter according to the invention, a light source for performing the optical analytical technique and a light analysis unit for analyzing light collected via the catheter as well as a power source for the ultrasound transducer (or pulsed light source in the case of optoacoustic stimulation) and wires/means for collecting and analyzing ultrasound signals from the target tissue. For example, for Raman spectroscopy, the system will include a light source such as a laser, for example, a feedback-stabilized multi-mode laser diode or a single-mode laser and a Raman spectrometer for measuring/analyzing light collected from a target tissue. For LIFS, the system will include a light source such as a laser and a fluorescence spectrometer. For TR-LIFS a spectrometer having temporal resolution may be used. For intereferometry, such as OCT, the system may include a broadband light source such as a superluminescent light-emitting diode or a pico-second pulse laser and an interferometer, such as a Michelson interferometer for analyzing light. One or more computers, or computer processors generally working in conjunction with computer accessible memory, may be part of the system for controlling the various elements and operations of the system and/or for analyzing information obtained by the system.

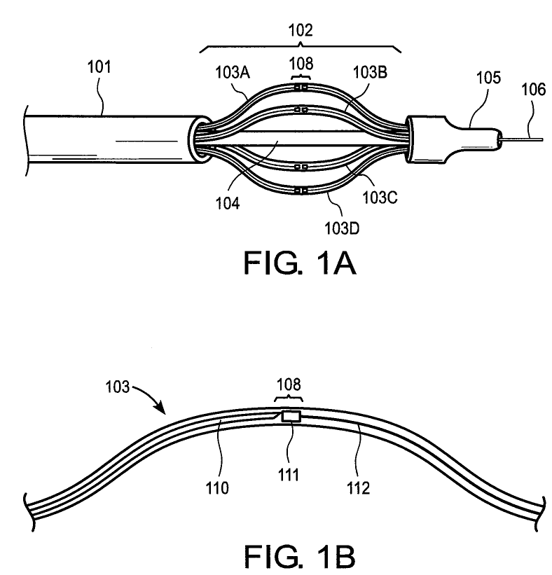

FIG. 1A shows a basket-style intravascular catheter comprising an outer shaft 101, a basket section 102 that includes radially extendable, wall-contacting/wall-approaching probe arms 103A-D each of which includes a side-viewing optical element for an optical analytical technique, such as Raman spectroscopy, and an ultrasound transducer for intravascular ultrasound. A central catheter shaft 104 runs through the center of basket section 102 and connects to distal tip 105 of the catheter. Central shaft 104 is hollow to receive a guide wire 106, which is shown extending out of distal tip 105.

Each of probe arms 103A-D is bowed or bowable outward as shown and includes a wall-contacting/wall-approaching portion 108 that is most radially extended/extendable. An optical fiber runs from the proximal end of the catheter up the proximal side of each probe arm and terminates in the wall contacting portion of the probe arm. The distal end of the optical fiber is angled to provide a lateral-viewing field. Distally adjacent to the distal viewing end of the optical fiber is an ultrasound transducer, such a high-frequency ultrasound transducer. In the embodiment shown, one or more wires are connected to the ultrasound transducer and run distally through the probe arm to enter the central shaft of the catheter via or near the distal tip of the catheter, after which they run to the proximal end of the catheter to connect to the ultrasound power source and analyzer unit. A reverse configuration of optical and ultrasound probe components is also provided by the invention. In either case, having the optical fiber(s) and ultrasound transducer wires enter from opposite ends of a probe arm minimizes the required cross-section dimension of the probe arm. However, the invention also provides that the optical fiber(s) and ultrasound transducer wire(s) may enter from the same side of a probe arm.

FIG. 1B shows the cross-sectional detail of a probe arm of the catheter embodiment of FIG. 1A. As shown, a side-viewing optical fiber 110 enters one end of the probe arm and terminates in the wall-contacting/wall-approaching portion 108 of the probe arm. Adjacent to the side-viewing portion of the optical fiber, in the wall-contacting/wall-approaching portion of the probe arm, is a side-looking ultrasound transducer 111. The wire(s) 112 of the ultrasound transducer enter the end of the probe arm opposite that where the optical fiber enters and run to the ultrasound transducer.

FIG. 1C shows the detail of a variation of a probe arm as shown in FIG. 1A in which the ultrasound transducer 111 is flush with the wall-contacting/wall-approaching surface of the probe arm and uncovered by the material of the body of the probe arm. In this manner, the material of the body of the probe arm cannot interfere with the transmission and receipt of ultrasound signals by the transducer.

FIG. 1D shows the detail of a variation of a probe arm as shown in FIG. 1A in which the ultrasound transducer 111 is recessed from the wall-contacting/wall-approaching surface of the probe arm and an acoustical window 113 is provided in the probe arm to accommodate the field-of-view of the ultrasound transducer. Optical window 113 may be empty or it may be at least partially filled with an acoustically transparent material or one with a similar acoustic impedance as tissues, such as polymethylpentene (TPX®).

FIG. 1E shows an embodiment of a probe arm configuration in which a prism 114 is provided to laterally deflect and receive optical signals (light) from one face of the prism and laterally deflect and receive ultrasound from the other face of the prism. Here, there is no lateral beam deflecting configuration of optical fiber 110 and IVUS element 113 is oriented toward the prism rather than radially outward toward the tissue.

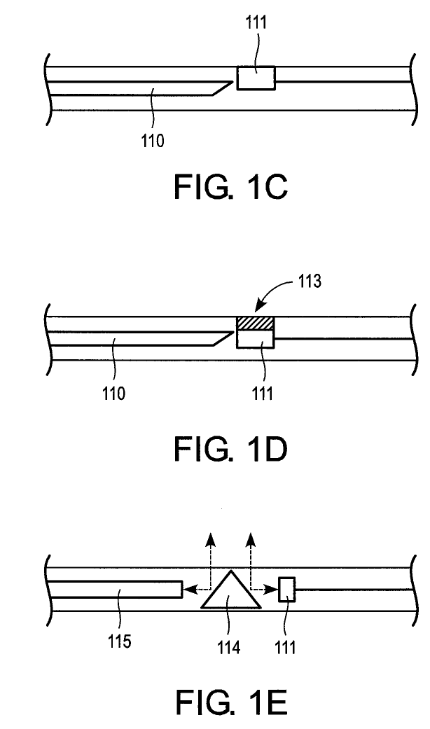

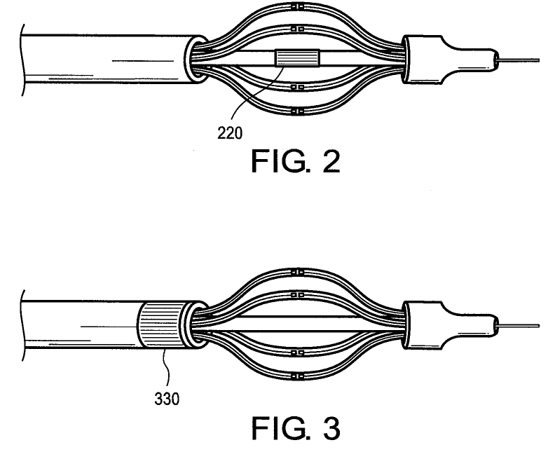

Optionally, the intravascular catheters of the invention may additionally include a centrally disposed (for example in/on a central shaft of the catheter) IVUS imaging element for radial scanning, such as found in conventional IVUS catheters. FIG. 2 shows an embodiment similar to that shown in FIG. 1 but further including a radially viewing IVUS element 220 centrally disposed on central shaft within the basket section of the catheter. FIG. 3 shows an embodiment similar to that shown in FIG. 1 but further including a radially viewing IVUS element 330 centrally disposed on the main (outer) shaft of the catheter, just proximal to the basket section. The invention also provides an embodiment (not shown) similar to that of FIG. 1 but having in addition a centrally disposed radially viewing IVUS element located distally of the basket section of the catheter, for example, in the proximal portion of the distal tip of the catheter. In embodiments having a centrally disposed radially viewing IVUS imaging element. the element may be of any type such as but not limited to a phase array IVUS imaging element or one involving mechanical rotation of the imaging element or of a radial acoustic deflector.

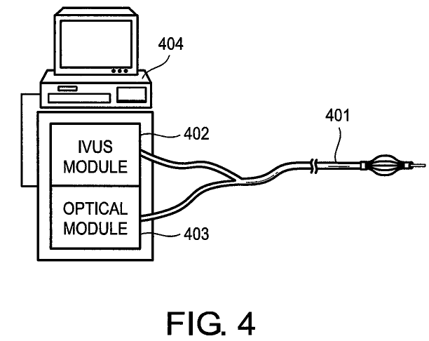

FIG. 4 schematically illustrates a diagnostic catheter system embodiment that includes a catheter 401 that includes one or more wall contacting IVUS probe elements and one or more wall-contacting/wall-approaching optical probe elements, such as the embodiment shown in FIG. 1, an IVUS module 402 including a power source for the ultrasound transducer and a ultrasound signal analyzer, an optical module 403 including a light source and a light measurement/analysis unit for analyzing collected light, and a computer 404 for controlling the components of the system and analyzing/presenting data obtained via the system. The system may also include a catheter pullback drive mechanism (not shown), such as those known in the art, so that IVUS and optical measurements may be obtained during a pullback procedure, in a blood vessel, such as an artery, for example, a coronary artery or carotid artery.

Raman spectroscopy has proven capable of determining the chemical composition of tissues and diagnosing human atherosclerotic plaques. Typical methods of collecting Raman scattered light from the surfaces of artery do not register information about how far the scattering element is from the collection optics. Two wavenumber regions that yield useful information for evaluating the condition of blood vessels are the so-called Raman fingerprint region i.e., approximately 200 to 2,000 cm⁻¹, and the so-called high wavenumber region, i.e., approximately 2,600 to 3,200 cm⁻¹. The collection of Raman spectra in the fingerprint (FP) region, through optical fibers is complicated by Raman “background” signal from the fibers themselves. In order to collect uncorrupted FP spectra, complicated optics and filters on the tips of catheters and often these designs require the use of multiple fibers. Since the Raman scattered signal is weak, large multimode fibers are utilized in the multi-fiber catheter designs, which creates an unwieldy catheter that is less than optimal for exploring delicate arteries, such as human coronary arteries. However, common optical fiber materials generate very little Raman background signal in the high wavenumber region, permitting a simplified, single optical fiber probe element implementation of intravascular Raman spectroscopy.

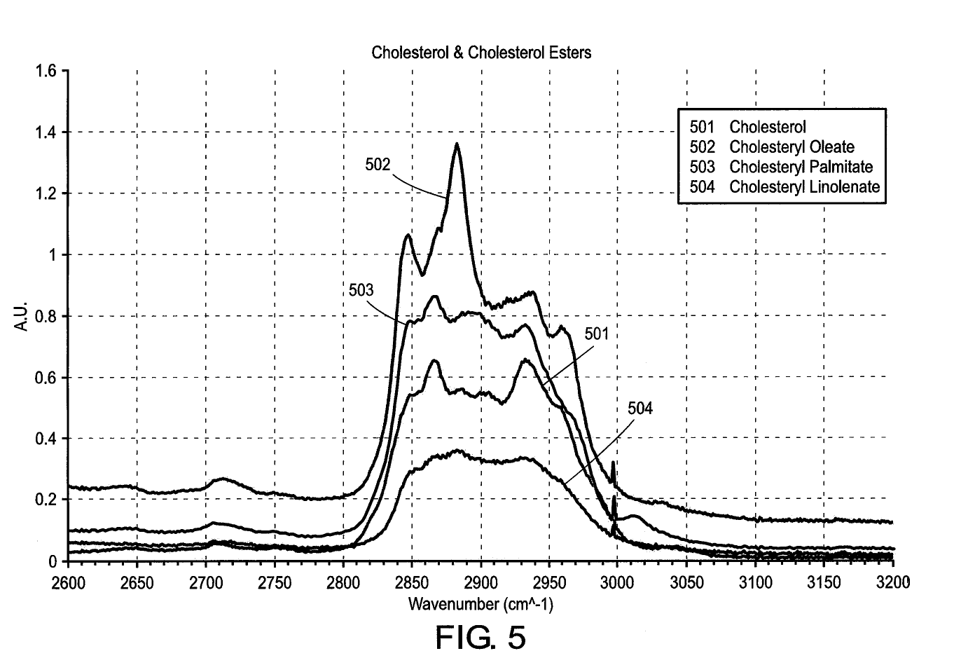

Since cholesterol and its esters have distinctive Raman scattering profiles within the Raman high wavenumber region, the use of the Raman high wavenumber region for analysis is particularly useful for locating and characterizing lipid-rich deposits or lesions as may occur in blood vessels, such a vulnerable plaques in arteries, such as in the coronary and carotid arteries. FIG. 5 shows Raman spectra of cholesterol and cholesterol esters in the high wavenumber region. Specifically, curve 501 is a Raman spectrum for cholesterol, curve 502 is a Raman spectrum for cholesteryl oleate, curve 503 is a Raman spectrum for cholesteryl palmitate and curve 504 is a Raman spectrum for cholesteryl linolenate.

One embodiment of the invention provides an intravascular catheter including at least one radially extendable wall-contacting/wall-approaching probe element that includes a wall-contacting/wall-approaching portion, which both includes a Raman spectroscopy probe element, such as a front or side viewing optical fiber assembly and an ultrasound transducer, such as a high-frequency ultrasound transducer operating at 20 MHz or above.

A related embodiment of the invention provides a basket-type intravascular catheter including a basket section that includes at least two radially extendable probe arms, each having a wall-contacting/wall-approaching portion, wherein at least one of the probe arms, such as all of the probe arms, include both a Raman spectroscopy probe element, such as a front or side viewing optical fiber assembly and an ultrasound transducer, such as a high-frequency ultrasound transducer operating at 20 MHz or above.

In embodiments in which a wall contacting probe arm includes both an IVUS element and an optical probe element, the IVUS and optical probe elements may be disposed closely adjacent to one another for close registration and/or overlap of their fields of view. In a variation of embodiments in which a wall contacting probe arm includes both an IVUS element and an optical probe element, either one or both of the IVUS element and optical probe element may be configured so that the field-of-view of one is diagonally incident on the field-of-view of the other.

A related embodiment of the invention provides a diagnostic catheter system for the evaluation of blood vessel walls that includes an intravascular diagnostic at least one radially extendable wall-contacting/wall-approaching probe element that includes a wall-contacting/wall-approaching portion, which includes both a Raman spectroscopy probe element, such as a front or side viewing optical fiber assembly and an ultrasound transducer, such as a high-frequency ultrasound transducer operating at 20 MHz or above, a light source such as a laser for stimulating Raman scattered light emissions from a target, a Raman spectrometer for analyzing Raman scattered light collected from a target, a power source for driving the ultrasound transducer and an ultrasound analyzer unit for receiving and analyzing the ultrasound signals from a sample. The system may be configured to collect and analyze Raman spectral data within the region of approximately 2,600 to 3,200 cm⁻¹, i.e., the so-called high wavenumber region, and/or the within the region of approximately 200 to 2,000 cm⁻¹, i.e., the so-called fingerprint region. The catheter may be a basket-style catheter including at least two probe arms, in which at least two probe arms include both a Raman spectroscopy probe element and an IVUS element. The Raman spectroscopic probe element may, for example, consist of a single optical fiber and the system configured to perform high wavenumber Raman spectroscopy via the single optical fibers of the probe arms. The intravascular ultrasound probe element may operate at a frequency of 20 MHz or higher, to provide high-resolution.

Advantageously, the system may be configured to provide depth-resolved chemical composition information about a target based on Raman spectroscopic data and intravascular ultrasound data obtained from interrogating the target using the intravascular diagnostic catheter. One embodiment of the invention utilizes Raman scattered light shifted in the high wavenumber (HW) region, i.e., approximately 2,600 to 3,200 cm⁻¹, and combines this information with IVUS data, such as from IVUS operating at frequencies of 10 MHz or greater, for example, high-resolution IVUS operating at 20 MHz or greater, to provide chemical compositional information as a function of depth in a lumen wall, such as a blood vessel wall, such as an artery wall.

In any of the embodiments having multiple wall-contacting/wall-approaching ultrasound elements, one or more multiplexers may be used to reduce the number of wires needed to carry signals down and out of the catheter for analysis. For basket catheter embodiments, the one or more multiplexers may, for example, be positioned in the distal tip of the catheter, if the lead wires to the ultrasound transducers run in the distal section of the probe arms, or just proximally of the basket section in the lead wires to the ultrasound transducers run in the proximal section of the probe arms.

The invention also generally provides methods for evaluating the condition of a blood vessel such as an artery, such as a human coronary or carotid artery, using an intravascular catheter and/or intravascular catheter system according to the invention to interrogate the wall of the vessel using IVUS alone or IVUS in combination with an optical analytical technique such as Raman spectroscopy. Atherosclerotic lesions and lipid rich deposits and/or lesions, such as vulnerable plaques, may be located and/or characterized in a blood vessel such as an artery by interrogating the blood vessel wall by IVUS alone or IVUS in combination with an optical analytical technique such as Raman spectroscopy using a catheter or catheter system according to the invention.

One embodiment of the invention provides a method for evaluating the wall of a blood vessel such an artery, such as a coronary or carotid artery, such as a human coronary or carotid artery, that includes the steps of:

providing a intravascular catheter including at least one radially extendable wall-contacting/wall-approaching probe element including an IVUS element, such as an ultrasound transducer operating at 10 MHz or above, for example, a high-frequency ultrasound transducer operating at 20 MHz or above;

disposing the wall-contacting/wall-approaching probe element of the catheter in a blood vessel; and

taking ultrasound readings of the vessel wall at one or more locations in the blood vessel using the IVUS element.

A related embodiment of the invention provides a method for evaluating the wall of a blood vessel such an artery, such as a coronary or carotid artery, such as a human coronary or carotid artery, that includes the steps of:

providing an intravascular basket catheter including a basket section that includes at least two radially extendable probe arms, such as 2, 3, 4, 5, 6, 7 or 8 probe arms, each having a wall-contacting/wall-approaching portion, wherein at least one of the probe arms, or at least two of the probe arms, or all of the probe arms, include a side viewing IVUS element, such as an ultrasound transducer operating at 10 MHz or above, for example, a high-frequency ultrasound transducer operating at 20 MHz or above;

disposing the basket section of the catheter in a blood vessel; and

taking ultrasound readings of the vessel wall at one or more locations in the blood vessel via the probe elements.

One embodiment of the invention provides a method for evaluating the wall of a blood vessel such an artery, such as a coronary or carotid artery, such as a human coronary or carotid artery, that includes the steps of:

providing a intravascular catheter including at least one radially extendable wall-contacting/wall-approaching probe arm including a side-viewing IVUS element, such as an ultrasound transducer operating at 10 MHz or above, for example, a high-frequency ultrasound transducer operating at 20 MHz or above, and a side viewing optical probe element;

disposing the probe arm in a blood vessel; and

taking both ultrasound readings and optical analytical readings of the vessel wall at one or more locations in the blood vessel via the probe arm(s).

The optical analytical readings may, for example, include measurement of Raman shifted light, for example, in the high wavenumber region and/or fingerprint region. The optical analytical reading may, for example, include fluorescence spectroscopy measurements, such as time-resolved fluorescence spectroscopy measurements.

A related embodiment of the invention provides a method for evaluating the wall of a blood vessel such an artery, such as a coronary or carotid artery, such as a human coronary or carotid artery, that includes the steps of:

providing an intravascular basket catheter including a basket section that includes at least two radially extendable probe arms, such as 2, 3, 4, 5, 6, 7 or 8 probe arms, each having a wall-contacting/wall-approaching portion, wherein at least one of the probe arms, or at least two of the probe arms, or all of the probe arms, include an optical probe element, such as a side-viewing optical fiber assembly and a IVUS element, such as an ultrasound transducer operating at 10 MHz or above, for example, a high-frequency ultrasound transducer operating at 20 MHz or above;

disposing the basket section of the catheter in a blood vessel; and

taking both ultrasound readings and optical analytical readings of the vessel wall at one or more locations in the blood vessel via the probe elements of the probe arms.

Again, the optical analytical readings may, for example, include measurement of Raman shifted light, for example, in the high wavenumber region and/or fingerprint region. The optical analytical reading may, for example, include fluorescence spectroscopy measurements, such as time-resolved fluorescence spectroscopy measurements.

It should be understood for the above methods that the probe arms are radially extended to contact or closely near the vessel walls in order to take the recited readings. Thus, the probe arms and in particular the portion including the IVUS viewing element and optical viewing element if any, may be configured to contact or approach the vessel wall. As used herein, the term “wall-approaching” means that the probe arm and the viewing portion thereof in particular is configured to near the vessel wall and/or contact the vessel wall. It will be readily recognized by those knowledgeable in the art that one or more probe arms may be in contact with a vessel wall at one time and not at another during the course of a procedure due to the changing geometry of a subject blood vessel and the present invention is intended to cover all such situations. The step of taking readings may include taking the recited readings at more than one longitudinal location in a blood vessel, for example, while the catheter is pulled back by operation of a catheter pullback mechanism.

Each of the patents and other publications cited in this disclosure is incorporated by reference in its entirety.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. An intravascular diagnostic catheter, comprising:
a proximal end and a distal insertion end; and
a basket section disposed at or near the distal insertion end, said basket section comprising at least two radially extendable wall-approaching probe arms each including a wall-approaching portion,
wherein each of at least two of the probe arms comprises a laterally-viewing ultrasound transducer within or near the wall-approaching portion of the probe arm.

2. The catheter of claim 1, wherein the catheter is sized and configured for evaluating human coronary arteries or human carotid arteries.

3. The catheter of claim 1, wherein the ultrasound transducer operates at 20 MHz or above.

4. An intravascular diagnostic catheter, comprising:
a proximal end and a distal insertion end; and
a basket section disposed at or near the distal insertion end, said basket section comprising at least two radially extendable wall-approaching probe arms each including a wall-approaching portion,
wherein each of at least two of the probe arms comprises a laterally-viewing high-frequency ultrasound transducer within or near the wall-approaching portion of the probe arm and a laterally-viewing optical element within or near the wall-approaching portion of the probe arm.

5. The catheter of claim 4, wherein the catheter is sized and configured for evaluating human coronary arteries or human carotid arteries.

6. The catheter of claim 4, wherein
the laterally viewing high-frequency ultrasound transducer is operably connected to at least one wire running from the proximal end of the catheter to the transducer; and
the laterally-viewing optical element is operably connected to at least one optical fiber running from the proximal end of the catheter to the optical element.

7. An intravascular diagnostic catheter, comprising:
a proximal end and a distal insertion end; and
at least one radially extendable probe arm disposed at or near the distal insertion end including a wall-approaching portion,
wherein the at least one radially extendable probe arm comprises a high-frequency ultrasound transducer within or near the wall-approaching portion of the probe arm and an optical probe element within or near the wall-approaching portion of the probe arm.

8. The catheter of claim 7, wherein the at least one radially extendable probe arm comprises at least two radially extendable probe arms.

9. The catheter of claim 7, wherein
the high-frequency ultrasound transducer is operably connected to at least one wire running from the proximal end of the catheter to the transducer; and
the optical probe element is operably connected to at least one optical fiber running from the proximal end of the catheter to the optical element.

10. The catheter of claim 9, wherein the at least one radially extendable probe arm comprises at least two radially extendable probe arms.

11. An intravascular interrogation system, comprising:
an intravascular catheter according to claim 7;
a power source operably connected to the ultrasound transducer;
an ultrasound signal analyzer;
a laser source in optical communication with the optical probe element; and
a Raman spectrometer in optical communication with the optical probe element.

13. The system of claim 11, wherein the at least one radially extendable probe arm comprises at least two radially extendable probe arms.

14. The system of claim 11, wherein the catheter is sized and configured for evaluating human coronary arteries or human carotid arteries.

15. The system of claim 11, wherein the catheter is a basket catheter comprising a basket section which comprises the probe arms.

16. The system of claim 15, wherein the catheter is sized and configured for evaluating human coronary arteries or human carotid arteries.

17. A method for evaluating the wall of a blood vessel, comprising the steps of:
providing an intravascular catheter including at least one radially extendable wall-approaching probe arm including an IVUS element;
disposing the wall-approaching probe element of the catheter in a blood vessel; and
taking ultrasound readings of the vessel wall at one or more locations in the blood vessel using the IVUS element.

18. The method of claim 17, wherein the ultrasound transducer operates at 20 MHz or above.

19. The method of claim 17, wherein intravascular catheter is an intravascular basket catheter comprising a basket section that comprises the at least one radially extendable wall-approaching probe arm.

20. The method of claim 19, wherein the ultrasound transducer operates at 20 MHz or above.

21. A method for evaluating the wall of a blood vessel, comprising the steps of:
providing an intravascular catheter including at least one radially extendable wall-approaching probe arm including a side-viewing IVUS element and a side-viewing optical probe element;
disposing the probe arm in a blood vessel; and
taking both ultrasound readings and optical analytical readings of the vessel wall at one or more locations in the blood vessel via the at least one probe arm.

22. The method of claim 21, wherein the ultrasound transducer is operates at 20 MHz or above.

23. The method of claim 21, wherein taking optical analytical readings comprises analyzing Raman scattered light collected from the one or more locations in the blood vessel.

24. The method of claim 23, comprising analyzing Raman scattered light in the high wavenumber region.

25. The method of claim 21, wherein intravascular catheter is an intravascular basket catheter comprising a basket section that comprises the at least one radially extendable wall-approaching probe arm.
